(19) United States
(12) Patent Application Publication
(10) Pub. No.: US 2006/0015130 A1
(43) Pub. Date: Jan. 19, 2006

(54) Catheter tunneler adapter

(75) Inventors: Earl Voorhees JR., Warrington, PA (US); Angela Wentling, Sassamansville, PA (US)

Correspondence Address: MONTE & MCGRAW, PC, 4092 SKIPPACK PIKE, P.O. BOX 650, SKIPPACK, PA, 19474, US

(73) Assignee: Medical Components, Inc., Harleysville, PA (US)

(21) Appl. No.: 11/180,138

(22) Filed: Jul. 13, 2005

Related U.S. Application Data
(60) Provisional application No. 60/587,679, filed on Jul. 14, 2004.

(51) Int. Cl. A61B 17/00

(52) U.S. Cl. 606/190

(57) ABSTRACT

A catheter tunneling assembly (100) used to tunnel a distal end of a multilumen catheter assembly (200) through a subcutaneous tunnel prior to inserting the catheter assembly into a patient. Adapter (130) facilitates connection of a trocar (110) with the catheter assembly and provides a smooth transition. The adapter has an inner member (130) that is affixed to the proximal end (114) of the trocar (110) and includes a proximal projection (134) that coextends along a distal portion of the catheter assembly (200); and an outer member (160) that is slidable from distally of the inner member to surround the proximal trocar end, the inner member (130) and a distal portion of the catheter assembly (200) pressing the proximal projection (134) of the inner member (130) into frictional engagement with a catheter portion. A method includes providing a tunneler assembly having a body (100,130) with a first and second extensions (126,134), and a slider (160); inserting the first extension into a catheter lumen; and sliding the slider (160) along the body toward the catheter to surround the tunneler assembly body and catheter end and bias the second extension (134) toward the first extension (126) to frictionally engage the catheter portion therebetween.

### CROSS-REFERENCE TO RELATED APPLICATION

This relates to and claims priority from Provisional U.S. Patent Application Ser. No. 60/587,679 filed Jul. 14, 2004.

### FIELD OF THE INVENTION

The present invention relates to a tunneler device for subcutaneously tunneling a catheter under a patient's skin prior to insertion into a patient's blood vessel.

### BACKGROUND OF THE INVENTION

Catheters for the introduction or removal of fluids may be located in various venous locations and cavities throughout the body of a patient for introduction of fluids to the body or removal of fluids from the body. Such catheterization may be performed by using a single catheter having multiple lumens. A typical example of a multiple lumen catheter is a dual lumen catheter in which one lumen introduces fluid and the other lumen removes fluid. An example of such a multiple lumen catheter is the SPLIT STREAM™ catheter, manufactured by Medical Components, Inc. of Harleysville, Pa.

Generally, to insert any catheter into a blood vessel, the vessel is identified by aspiration with a long hollow needle in accordance with the well-known Seldinger technique. When blood enters a syringe attached to the needle, indicating that the vessel has been found, a thin guide wire is then introduced, typically through a syringe needle or other introducer device into the interior of the vessel. The introducer device is then removed, leaving the end portion of the guide wire that has been inserted into the vessel within the vessel and the opposing end of the guide wire projecting beyond the surface of the skin of the patient. At this point, several options are available to a physician for catheter placement. The simplest is to pass a catheter into the vessel directly over the guide wire. The guide wire is then removed, leaving the catheter in position within the vessel. However, this technique is only possible in cases where the catheter is of a relatively small diameter, made of a stiff material, and not significantly larger than the guide wire. For example, this technique may be used to insert small diameter dual lumen catheters into a patient. If the catheter to be inserted is significantly larger than the guide wire, a dilator and sheath assembly is passed over the guide wire to enlarge the hole. The guide wire and dilator are then removed, and the catheter is then inserted through the sheath and into the vessel. The sheath is then removed by peeling the sheath from around the catheter while pulling the sheath from the vessel.

For chronic catheterization, in which the catheter is intended to remain inside the patient for an extended period of time, such as for weeks or even months, it is typically desired to subcutaneously tunnel the catheter using various tunneling techniques. The catheter is typically tunneled into the patient prior to inserting the catheter into the patient's vein.

### SUMMARY OF THE INVENTION

The present invention comprises a catheter tunneler assembly used to tunnel a distal end of a multilumen catheter assembly through a subcutaneous tunnel prior to inserting the catheter assembly into a patient. A distal end of the assembly includes a tunneler, such as a trocar, and an adapter that facilitates connection of the trocar with the catheter assembly and provides a smooth transition between the trocar's proximal end and the catheter assembly. The adapter is also adapted to restrict movement of the catheter assembly away from the tunneling assembly. The adapter comprises an inner member having a trocar gripping section and a catheter engagement section, and an outer member slidable from distally of the inner member to a position surrounding the inner member and an end portion of the catheter assembly. The outer or sheath member is adapted to press the adapter inner member projection toward the tunneler projection so that a catheter lumen wall is frictionally held therebetween.

In a preferred embodiment, the adapter inner member is affixed to the proximal end of the tunneler in a manner exposing a projection of the tunneler for insertion into a first lumen of the catheter assembly, and further includes a projecting portion coextending along and spaced from the tunneler projection to coextend along and adjacent to an inner, second lumen of the catheter assembly. The outer member is adapted to be slid over the adapter inner member and over both the tunneler projection after being positioned within the first lumen of the catheter assembly, and the adapter projection that extends along and adjacent the catheter assembly inner lumen, so that a proximal portion of the outer adapter member extends beyond the inner member's adapter projection to surround an adjacent end portion of the catheter assembly.

The present invention also includes a method for connecting a catheter assembly to a tunneling assembly, having the steps of: providing a catheter having at least one lumen; providing a catheter tunneler assembly having a proximal end body, a first extension, a second extension spaced laterally from the first extension, and a slider; inserting the first extension into the at least one lumen of the catheter; and, sliding the slider along the body toward the catheter to surround the proximal end body and the first and second extensions and a distal catheter end portion to bias the second extension toward the first extension into frictional engagement with a catheter portion therebetween.

### BRIEF DESCRIPTION OF THE DRAWINGS

The accompanying drawings, which are incorporated herein and constitute part of this specification, illustrate the presently preferred embodiment of the invention, and, together with the general description given above and the detailed description given below, serve to explain the features of the invention. In the drawings:

FIG. 1 is a side profile view of a catheter tunneler assembly comprised of a catheter tunneler and adapter according to an embodiment of the present invention;

FIG. 2 is a side profile view of the catheter tunneler shown in FIG. 1;

FIG. 3 is an enlarged end view of the catheter tunneler assembly shown in FIG. 1;

FIG. 4 is a side profile view of the adapter of the catheter tunneler assembly shown in FIG. 1;

FIG. 5 is an enlarged end view of the adapter taken along lines 5-5 of FIG. 4;

FIG. 6 is an enlarged view of the proximal end of the adapter shown in FIG. 5;

FIG. 7 is a sectional view of the tunneler assembly taken along line 7-7 of FIG. 1;

FIG. 8 is and end view of a tunneler grasper sheath for use with the tunneler assembly;

FIG. 9 is a longitudinal sectional view of the tunneler grasper sheath taken along line 9-9 of FIG. 8;

FIG. 10 is a side profile view of the catheter tunneler assembly of FIG. 1, connected to the distal end of a catheter; and

FIG. 11 is an enlarged perspective view, in section, of the tunneler assembly and grasper sheath connected to the distal end of a catheter.

### DETAILED DESCRIPTION OF THE INVENTION

In the drawings, like numerals indicate like elements throughout. Certain terminology is used herein for convenience only and is not to be taken as a limitation on the present invention. When describing or referring to the catheter tunneler adapter, the words “proximal” and “distal” refer to directions away from and closer to, respectively, the pointed tip of the trocar that makes up a portion of the catheter tunneling assembly according to the present invention. When describing or referring to a catheter, the words “proximal” and “distal” refer to directions away from and closer to, respectively, the tip of the catheter that is inserted in the blood vessel closest to the patient's heart. The terminology includes the words above specifically mentioned, derivatives thereof, and words of similar import. The following describes a preferred embodiment of the invention. However, it should be understood based on this disclosure, that the invention is not limited by the preferred embodiment described herein.

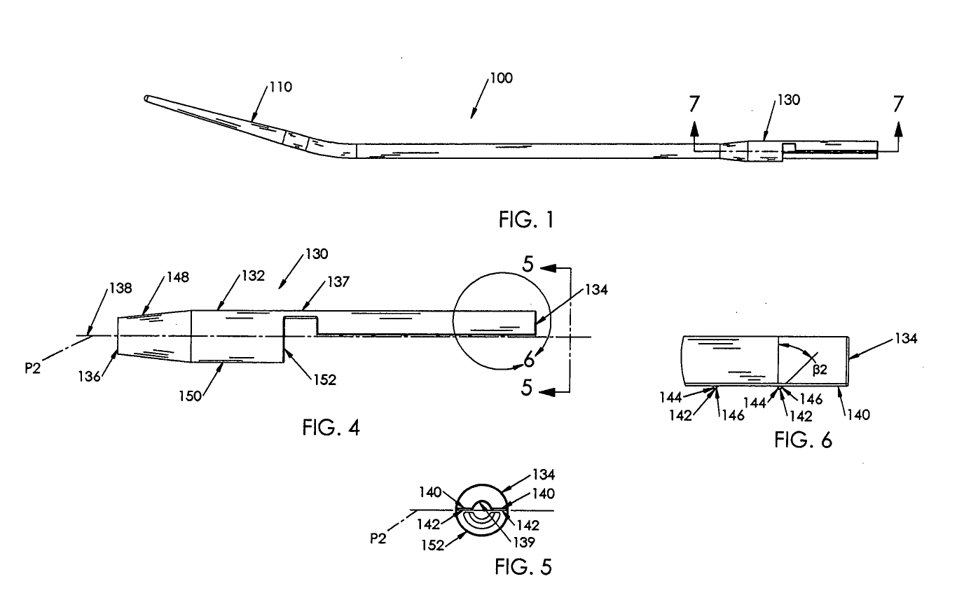

Referring to FIG. 1, a side profile view of a catheter tunneling assembly 100 according to a preferred embodiment of the present invention is shown. Preferably, the catheter tunneling assembly 100 is used to tunnel a distal end of a multilumen catheter assembly through a subcutaneous tunnel prior to inserting the catheter assembly into the patient. Those skilled in the art will recognize that the assembly 100 may be used to tunnel the proximal end of a multilumen catheter, or either end of a single lumen catheter, without departing from the scope of the present invention.

The tunneling assembly 100 includes a distal portion, or tunneler, such as a trocar 110 and an adapter having an inner body or gripper 130 and an outer member or slider or grasping sheath 160 that can be disposed over the body. The adapter inner body 130 facilitates connection of the trocar 110 with the catheter assembly and provides a smooth transition between a proximal end 114 of the trocar 110 and the catheter assembly to minimize tearing or snagging of subcutaneous tissue during catheter tunneling. Additionally, the adapter serves to restrict the movement of the catheter assembly away from the tunneling assembly 100.

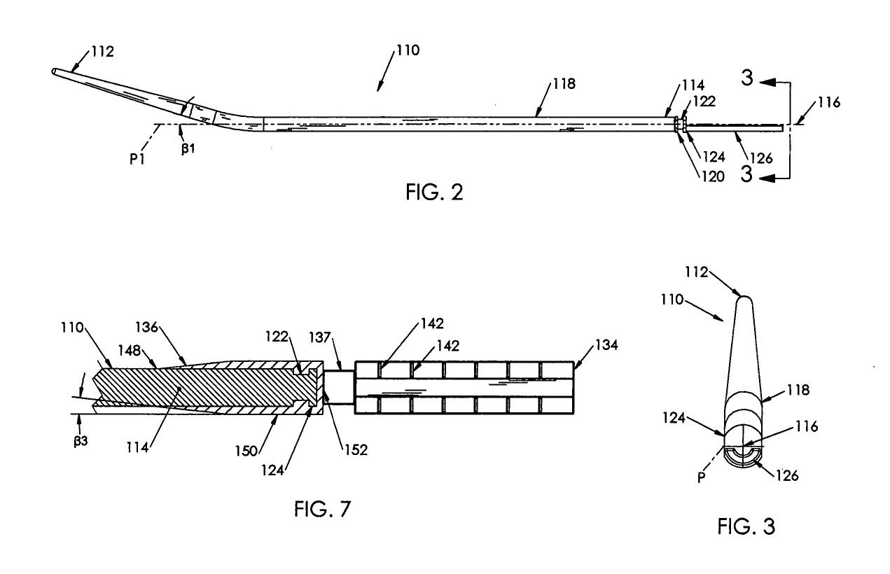

Referring now to FIG. 2, the trocar 110 includes a distal tip 112, the proximal end 114, and a longitudinal axis 116 extending through the proximal end 114. A longitudinal plane “P1” extends along the longitudinal axis 116 perpendicular to the plan of the paper containing FIG. 2. An elongated body 118 extends between the distal tip 112 and the proximal end 114. The body 118 is preferably approximately 3.7 mm in diameter and tapers to a distal tip 112 that may be either blunt or sharp. The body also preferably bends at a predetermined location along the length of the body 118. As shown in FIG. 2, the body 118 is bent at an angle β1 of approximately 16 degrees, although those skilled in the art will recognize that the body 118 may be bent more or less than 16 degrees or have no bend at all. Preferably, the trocar 110 is of unitary construction and is preferably constructed from 303 stainless steel, although those skilled in the art will recognize that the trocar 110 may be constructed from other suitable materials.

The proximal end 114 includes a tapered ring 120 that increasingly tapers in a proximal direction from a diameter of the body 118 to a slightly larger diameter, such as approximately 0.5 mm, than that of the body 118. A circumferential channel 122 is disposed distally of the ring 120. The channel 122 is used to positively secure the adapter 130 to the trocar 110, as will be explained in more detail later herein. A locking ring 124 is disposed proximally of the channel 122. Preferably, the distal ring 124 is the same diameter as the tapered ring 120.

A first projection or catheter insert prong 126 is disposed proximally of the distal ring 124. As seen in FIG. 3, the insert prong 126 has a generally C-shaped or semi-annular profile and is disposed on one side of the longitudinal plane P1. The insert prong 126 is shown to be configured to fit into the distal tip of a lumen of a multi-lumen catheter, such as the TORRENT FLOW™ catheter manufactured by Medical Components, Inc. of Harleysville, Pa. Although insert prong 126 is shown as generally C-shaped in profile, those skilled in the art will recognize that any shape suitable to be inserted into the distal tip of a catheter lumen may be used.

Referring to FIG. 4, the adapter inner body 130 includes a generally elongated body 132 having a proximal portion 134 and a distal portion or second projection 136. A connector or hinge 137 connects the proximal portion 134 and the distal portion 136. The connector 137 has a smaller cross-sectional size than the proximal portion 134 or the distal portion 136 to provide some flexibility between the proximal portion 134 and the distal portion 136.

A longitudinal axis 138 extends through the body 132 between the proximal portion 134 and the distal portion 136. A longitudinal plane “P2” extends along the longitudinal axis 138 perpendicular to the plan of the paper containing FIG. 4. The proximal portion 134 extends wholly on one side of the longitudinal plane P2, and preferably it is angled slightly away from plane P2 to facilitate insertion of the tunneler assembly onto the catheter end. As seen in FIG. 5, the proximal portion 134 has a generally semi-annularly shaped cross-section, with an inwardly directed surface having a curved face 139 and generally flat elongated longitudinal portions 140 on either side of the curved face 139.

As seen in FIG. 6, each longitudinal portion 140 includes a plurality of ribs 142 that extend transverse to the length of the proximal portion 134. Each rib 142 has a distal face 144 that extends perpendicular to the longitudinal portion 140 and a proximal face 146 that extends at an angle β2 from the perpendicular. Preferably, P2 is approximately 45 degrees, although those skilled in the art will recognize that β2 may be more or less than 45 degrees.

Referring to FIGS. 4 and 7, the distal portion 136 includes a hollow tapered distal end 148 and a generally hollow cylindrical body 150. The taper of the distal end 148 is preferably approximately 12 degrees. As seen in FIG. 7, the proximal end 152 of the body 150 is closed.

Adapter body 130 is constructed from HDPE and is overmolded over the proximal end 114 of the trocar 110. The overmold process disposes HDPE into the channel 122, as seen in FIG. 7. The HDPE within the channel 122 provides a positive lock of the adapter inner body 130 onto the proximal end 114 of the trocar 110 so that the adapter inner body will not be separated from the trocar 110 during catheter tunneling. The overmold process also forms the distal end 136 of the adapter inner body with a taper having a taper angle β3 of approximately 12 degrees.

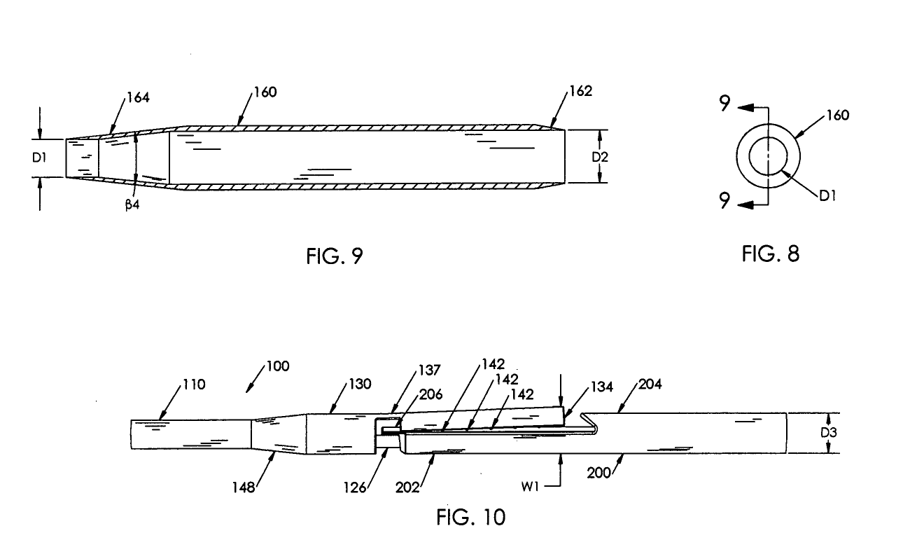

Referring now to FIGS. 8 and 9, an adapter slider or outer member or grasper sheath 160 is shown. As seen in FIG. 9, the grasper sheath 160 is generally tubular in shape, with a tapered proximal end 162 and a tapered distal end 164. Preferably, the distal end 164 is tapered at an angle β4 that is equivalent to angle β3. Grasper sheath 160 is sized to allow the trocar 110 and the adapter inner body 130 to be inserted through its proximal end 162 and pulled through the distal end 164 having an inner diameter D1. Preferably, the inner diameter D2 at proximal end 162 is dimensioned to match the catheter's outer diameter. Preferably, also, the grasper sheath 160 is constructed from polypropylene.

In use, the tunneling assembly 100 is engaged with the distal end of a catheter 200, as shown in FIG. 10. The catheter 200 has a first lumen 202, a second lumen 204, and a third or inner lumen 206 and has an outer diameter D3. Such a catheter 200 is the TORRENT FLOW™ catheter. In cross-section, the lumens 202, 204 are each generally C-shaped and juxtaposed from each other across a plane, and the third lumen 206 is generally circular and is centered between the first and second lumens 202, 204. The catheter insert prong 126 is inserted in the distal end of the first lumen 202 until the distal end of the third lumen 206 engages the proximal end 152 of the hollow cylindrical body 150 of the adapter body 130. The curved face 139 of the proximal portion 134 is sized and shaped to fit over the exterior of the third, inner lumen 206 distal of the second lumen 204. Connector 137 preferably is shaped to dispose the proximal end 134 of the adapter 130 slightly away from the catheter 200, and the overall width of the assembly at the tip of proximal end 134, including the catheter 200, is designated as W, which is greater than D3. Inner diameter D2 of outer adapter member at its proximal end 162, as stated hereinabove, is less than W.

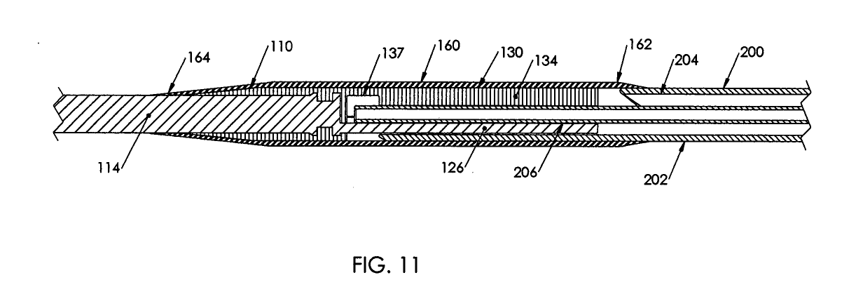

After the tunneling assembly 100 is inserted onto the distal end of the catheter 200, the distal tip 112 of the trocar 110 is inserted into the proximal end 162 of the grasper sheath 160, through the grasper sheath 160 and out the distal end 164 of the grasper sheath 160. The grasper sheath 160 is slid proximally along the trocar 110 and the adapter inner body 130 until the tapered distal end 164 of the grasper sheath 160 engages its tapered distal end 148. As shown in FIG. 11, the proximal end 162 of the grasper sheath 160 engages the proximal end 134 of the adapter inner body 130 and pivots the proximal end 134 of the adapter inner body toward the catheter to be biased against the exterior of the first lumen 202 so that the ribs 142 at least slightly dig into the exterior of the first lumen 202 to provide a secure engagement of the adapter body 130 with the catheter 200.

After the tunneling assembly 100 is secured to the catheter 200 as described above, the catheter 200 is tunneled according to known procedures. After tunneling, the grasper sheath 160 is slid distally along the trocar 110 until the proximal end 134 of the adapter inner body 130 is freed from the grasper sheath 160. The proximal end 134 then again is angled by the connector 137 to space the ribs 142 from the exterior of the first lumen 202 to facilitate disassembly of the tunneler from the catheter. The catheter insert prong 126 is removed from the distal end of the catheter 200 and the tunneling assembly 100, with grasper sheath 160, may be discarded.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A catheter tunneler assembly comprising:
an elongated tunneler comprising a first portion and a second portion, wherein the second portion has a generally circular end and an extension extending from the second portion in a direction generally away from the first portion;
a gripper having a first gripping portion and a second gripping portion; and
a generally tubular slider;
wherein the first gripping portion is adapted to engage the generally circular end of the elongated tunneler;
wherein the second gripping portion extends from the first gripping portion parallel to the extension of the tunneler;
wherein the slider is adapted to be slid longitudinally along the elongated tunneler and the gripper; and
wherein the slider is adapted to bias the second gripping portion towards the extension when the slider is disposed about the second gripping portion and the extension.

2. The catheter tunneler assembly according to claim 1, wherein the extension is adapted to be inserted into a catheter lumen.

3. The catheter tunneler assembly according to claim 2, wherein the extension is generally semi-annular in cross-section.

4. The catheter tunneler assembly according to claim 1, wherein the second gripping portion and the extension are adapted to engage at least a portion of a catheter lumen between the second gripping portion and the extension.

5. The catheter tunneler assembly according to claim 1, wherein the second gripping portion is semi-annular.

6. The catheter tunneler assembly according to claim 1, wherein the second gripping portion comprises at least one rib on an inwardly facing surface.

7. The catheter tunneler assembly according to claim 1, wherein the second gripping portion is hingedly connected to the first gripping portion.

8. The catheter tunneler assembly according to claim 1, wherein the inner diameter of the proximal end of the slider is just less than the outer diameter of the tunneler assembly at second gripping portion and the extension at the catheter end.

9. The catheter tunneler assembly according to claim 1, wherein the gripping portion is overmolded onto the proximal end of the tunneler.

10. A catheter tunneler assembly comprising:
a generally elongated body portion having a first end and a second end;
a movable slider adapted to slide longitudinally along the generally elongated body portion; and
first and second extensions disposed on the second end and extending away from the first end;
wherein the first extension is adapted to be inserted into a catheter lumen;
wherein the second extension is adapted to be disposed outside of the catheter lumen when the first extension is inserted into the catheter lumen;
wherein the slider is adapted to frictionally engage the second extension when the slider is slid in a direction from the first end towards the second end; and
wherein the second extension is biased towards the first extension when the slider frictionally engages the second extension.

11. The catheter tunneler assembly according to claim 10, wherein the first extension has a generally semi-annular cross-section.

12. The catheter tunneler assembly according to claim 10, wherein the second extension has a generally semi-annular cross-section.

13. The catheter tunneler assembly according to claim 10, wherein at least one of the first and second extensions are hingedly connected to the second end.

14. A catheter tunneling assembly comprising:
a tunneler having:
a generally elongated tunneler body;
a first end;
a second end; and
a catheter engagement portion extending from the second end away from the tunneler body, wherein the catheter engagement portion is configured to be inserted into a catheter lumen;
a tunneler adapter having:
a generally elongated adapter body;
a first end including means for engaging the second end of the tunneler; and
a second end configured to frictionally engage the catheter lumen; and
a grasper sheath slidably disposable over tunneler and the tunneler adapter such that the grasper sheath biases the second end against an adjacent wall of the catheter lumen.

15. A method for connecting a catheter to a tunneling assembly comprising the steps of:
(a) providing:
a catheter having at least one lumen; and
a catheter tunneler assembly having a proximal end body, a first extension, a second extension spaced laterally from the first extension, and a slider;
(b) inserting the first extension into the at least one lumen of the catheter; and
(c) sliding the slider along the body towards the catheter so that it surrounds the tunneler assembly proximal end body and the first and second extensions and the catheter distal end and biases the second extension towards the first extension and into frictional engagement with a wall portion of the catheter therebetween.
